(19) United States
(12) Patent Application Publication
(10) Pub. No.: US 2020/0392538 A1
(43) Pub. Date: Dec. 17, 2020

(54) ITERATIVE GENOME ASSEMBLY

(71) Applicant: PRESIDENT AND FELLOWS OF HARVARD COLLEGE, Cambridge, MA (US)

(72) Inventors: George M. CHURCH, Cambridge, MA (US); Nili OSTROV, Cambridge, MA (US); Matthieu Marie LANDON, Cambridge, MA (US)

(73) Assignee: President and Fellows of Harvard College, Cambridge, MA (US)

(21) Appl. No.: 16/643,111

(22) PCT Filed: Aug. 29, 2018
(86) PCT No.: PCT/US2018/048418

§ 371 Date: Feb. 28, 2020

Related U.S. Application Data
(60) Provisional application No. 62/552,209, filed on Aug. 30, 2017.

(51) Int. Cl. C12N 15/90; C12N 15/65; C12N 15/10; C12N 15/11; C12N 9/22

(52) U.S. Cl. CPC C12N 15/902; C12N 15/65; C12N 2310/20; C12N 15/111; C12N 9/22; C12N 15/102

(57) ABSTRACT

Provided herein are methods for hierarchical genome assembly using nuclease-assisted homologous recombination, which enable scarless and iterative replacement of wild-type DNA with large (e.g., at least 50 kilobases (kb)) synthetic DNA segments at desired genomic loci.

### RELATED APPLICATIONS

This application claims the benefit under 35 U.S.C. § 119(e) of U.S. provisional application No. 62/552,209, filed Aug. 30, 2017, which is incorporated by reference herein in its entirety.

### FEDERALLY SPONSORED RESEARCH

This invention was made with government support under Contract No. DE-FG02-02ER63445 awarded by U.S. Department of Energy. The government has certain rights in the invention.

### BACKGROUND

Recombineering is a recombination-mediated genetic engineering technique based on homologous recombination systems in Escherichia coli (E. Coli), mediated by bacteriophage proteins, such as RecE, RecT and Gam from Rac prophage or Gam, Exo and Beta from bacteriophage lambda. While these recombineering systems are widely used in E. Coli for recombineering of synthetic (e.g., mutagenic) nucleic acids, there efficiency significantly decreases when using large DNA segments (e.g., at least 50 kilobase (kb) segments) as synthetic DNA (H. H. Wang et al. Nature. 460, 894-898 (2009)).

### SUMMARY

Provided herein, in some embodiments, are methods for hierarchical assembly of synthetic genomes using nuclease-assisted homologous recombination, which enable scarless and iterative replacement of wild-type DNA with large (e.g., at least 50 kb) synthetic DNA segments at desired genomic loci. These large segments are seamlessly integrated to construct a complete chromosome, for example. The nuclease is used to initiate and enhance DNA homologous recombination by creating targeted double-strand breaks in a segment of the parental genome. The double-strand breaks induce homologous recombination of a donor DNA segment (e.g., located episomally) with the parental genomic segment, which share long overlapping homology arm sequences (e.g., having a length of greater than 50 bp). The recombination event is catalyzed by a recombinase system (e.g., lambda phage recombineering system).

Advantageously, the methods of the present disclosure avoid recombination crossover, which can impede full integration of donor DNA. With current methods, direct exchange of genomic DNA with donor DNA often results in multiple crossover events when using donor DNA sequences that are highly similar to genomic sequence (such as shuffled, mutated, or recoded E. coli DNA). With the methods as provided herein, if the incoming donor DNA segment is similar to the genomic segment, the genomic segment is first replaced by a heterologous segment (e.g., selectable marker gene) through homologous recombination, and then incoming donor DNA is integrated through nuclease-assisted homologous recombination. No homology exists between donor DNA segment and heterologous DNA segment (excluding the homology arms flanking each segment), thus no crossover occurs, and the intact donor DNA segment is fully integrated.

Further, unlike current methods that than requiring both positive selection for the presence of the donor DNA segment and negative selection for the loss of a genomic marker(s) to validate successful integration, the methods of the present disclosure select only for the presence of the nuclease, obviating the need for multiple selectable markers. These methods are thus scarless, with no selectable markers remaining in the genome of a cell at the end of each cycle of assembly (e.g., addition of each donor DNA segment).

### BRIEF DESCRIPTION OF THE DRAWINGS

FIG. 1: Overview of scarless assembly of recoded genome. (1) Parental strain carries RNA-guided nuclease (e.g., Cas9) with gRNAs (yellow), a recombinase system (orange, e.g., lambda-red), a selectable marker gene (e.g., ZEOCIN™ resistance gene) at desired integration loci (grey), and a donor DNA segment (e.g., a plasmid containing recoded DNA 1). (2) gRNA targets cutting of the selectable marker gene in the genome of the parent cell to initiate and enhances recombination. (3) Recombination of homologous sequences between donor and acceptor is mediated by recombinase (e.g. lambda-red). (4) The resulting parental cell carries only the donor DNA segment on the genome. The process can be iterated multiple times by repeating with additional donor DNA segments at any desired genomic loci. A ‘reset’ step is used for each assembly cycle to introduce a selectable marker and the new donor DNA segment into the genomic loci.

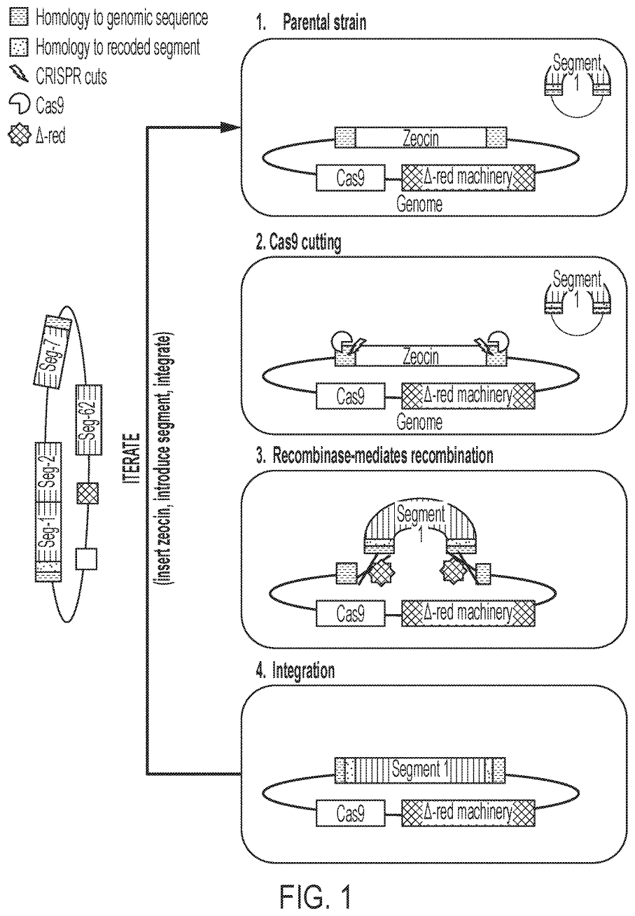

FIG. 2: Colony survival after Cas9 cutting, using 50 bp homology between donor and parental DNA. A single Cas9 cut site was tested in the target loci (‘zeo-1-cut’ represents a single cut site in the ZEOCIN™ resistance gene). A control sequence that should not be cut by Cas9 is also presented (‘attP-1-cut’). functional Cas9 selection works in parental cells containing Cas9 target (e.t. a ZEOCIN™ resistance gene), but not in wild-type (WT) E. coli. However, none of the resulting colonies show genomic integration of the recoded donor DNA segment or loss of the ZEOCIN™ resistance gene, indicating these conditions (e.g. 50 bp homology size, single Cas9 cut site only) were insufficient for successful recombination.

FIG. 3: Colony survival after Cas9 cutting, using 250 bp homology between donor and parental. multiple Cas9 cut sites in the target loci are tested. (‘zeo-v1’ and ‘zeo-v2’ represent two gRNAs with two different cut sites in ZEOCIN™ resistance gene; ‘zeo-I-F’ represent one gRNA with two cut sites at the sides of the zeocin gene). A control sequence that should not be cut by Cas9 is also presented (‘attP-1-cut’). Several of the resulting colonies show genomic integration of the recoded DNA segment or loss of the ZEOCIN™ resistance gene, indicating these conditions (250 bp homology size, multiple Cas9 cut sites) were sufficient for successful recombination.

### DESCRIPTION

The present disclosure provides methods and compositions (e.g., cells, genetic constructs, and kits) for targeted scarless integration of large DNA segments (e.g., at least 50 kb) from a donor into a receiver (parental) strain genome. Each assembly cycle (integration of a single segment) of these methods for genome assembly can be iterated for integration of multiple donor DNA segments in a sequential or parallel manner. Donor DNA segments may be assembled on a plasmid from synthetic DNA segments, for example. The donor DNA contains long overlapping homology sequences (e.g., longer than 50 base pairs (bp)) with the targeted insertion locus. In some embodiments, the targeted insertion locus is first replaced with a heterologous DNA segment, such as a selectable marker gene, to avoid undesired recombination. If the DNA is meant to replace a certain genomic segment (e.g., for genome recoding), it may be functionally validated prior to the replacement (N. Ostrov et al. Science. 353, 819-822 (2016)). The donor DNA is then introduced into the parental cells (e.g., via transformation (e.g., chemical transfection or electroporation), conjugation (e.g., via conjugative plasmids), or transduction (e.g., via bacteriophage)), which carry an inducible recombineering system (e.g., lambda-red, genomically integrated or on a plasmid). Cells are induced for expression of the selected recombineering system proteins (e.g., Gam, Exo and Beta), which mediate recombination of homologous sequences (referred to as homology arms or sequences).

In some embodiments, a sequence-specific nuclease (e.g., a restriction endonuclease or a programmable nuclease) that cleaves specifically at the genomic loci for integration is introduced in the cells (e.g., by plasmid transformation), before, during or after induction (activation) of expression of the recombineering system. In such embodiments, the nuclease may be constitutively expressed. The nuclease introduces double-strand breaks at that locus (or loci) of interest to enhance homologous recombination by the recombineering system and selectively destroying all cells where the recombination locus (or loci) has not been modified by the introduction of the donor DNA segment. In other embodiments, the parental cells are engineered to carry an inducible sequence-specific nuclease (e.g., genomically), thus, nuclease activity may be induced before, during or after induction of expression of the recombineering system.

In other embodiments, a RNA-guided nuclease (e.g., Cas9) and guide RNA (gRNA) targeting the genomic loci for integration are introduced in the cells (e.g., by plasmid transformation), before, during or after induction of expression of the recombineering system. In such embodiments, the RNA-guided nuclease and the gRNA may be constitutively expressed. In yet other embodiments, the parental cells are engineered to carry a RNA-guided nuclease (e.g., genomically), thus, only the gRNA targeting the genomic loci for integration is introduced before, during or after induction of expression of the recombineering system.

Cells are then allowed a selection-free recovery period (e.g., 3 hours to overnight in LB) and plated on selective plates for the sequence-specific nuclease component only. Colonies may then be screened for integration of the donor DNA segment into the parental genome at the desired locus (or loci) by PCR and whole-genome sequencing, for example.

Thus, provided herein, in some aspects, are methods that include (a) introducing into a parental cell a donor DNA segment flanked by first homology sequences having a length of longer than 50 nucleotide base pairs, wherein the parental cell comprises a genomic locus of interest flanked by second homology sequences homologous to the first homology sequences, (ii) an inducible recombineering system, and (iii) a sequence-specific nuclease that cleaves the genomic locus of interest; (b) inducing activity of the sequence-specific nuclease; and (c) inducing expression of the inducible recombineering system.

Also provided herein, in some aspects, are methods that include (a) introducing into a parental cell a donor DNA segment flanked by first homology sequences having a length of longer than 50 nucleotide base pairs, wherein the parental cell comprises a genomic locus of interest flanked by second homology sequences homologous to the first homology sequences, and (ii) an inducible recombineering system; (b) introducing into the cell a sequence-specific nuclease that cleaves the genomic locus of interest; and (c) inducing expression of the inducible recombineering system.

Other aspects of the present disclosure provide methods that include (a) introducing into a parental cell a donor DNA segment flanked by first homology sequences, wherein the parental cell comprises (i) a selectable marker gene integrated genomically and flanked by second homology sequences homologous to the first homology sequences, and (ii) an inducible recombineering system; (b) introducing into the parental cell (i) a RNA-guided nuclease or a nucleic acid encoding a RNA-guided nuclease and (ii) at least one nucleic acid encoding at least one guide RNA (gRNA) targeting the selectable marker gene; and (c) inducing expression of the inducible recombineering system.

Still other aspects of the present disclosure provide methods that include (a) introducing into a parental cell a donor DNA segment flanked by first homology sequences, wherein the parental cell comprises (i) a selectable marker gene integrated genomically and flanked by second homology sequences homologous to the first homology sequences, (ii) an inducible recombineering system, and (iii) a nucleic acid encoding a RNA-guided nuclease; (b) introducing into the parental cell a nucleic acid encoding a guide RNA (gRNA) targeting the selectable marker gene; and (c) inducing expression of the inducible recombineering system.

In some embodiments, the methods further comprise repeating steps (a)-(c) using a DNA segment having a sequence that is different from the DNA segment of step (a). In some embodiments, the methods further comprise repeating steps (a)-(c) multiple times, each time using a DNA segment having a sequence that is different from any other DNA segment introduced into the parental cell. For example, the methods as provided herein may be used to assemble different DNA segments of an entire genome or a portion of a genome.

The present disclosure also provides composition and kits comprising any one or more of the foregoing components of the methods.

It should be understood that any of the following aspects and embodiments described herein, including those only disclosed in the Examples section or any other single section of the specification, may combine with any other aspect(s) and/or embodiment(s), unless explicitly disclaimed.

### Parental Cells

A parental cell may be any cell into which exogenous DNA (e.g., recombinant or synthetic DNA) is introduced. Thus, a parental cell may be a eukaryotic cell (e.g., mammalian cell, plant cell or fungal cell) or a prokaryotic cell (e.g., bacterial cell). In some embodiments, a parental cell is a bacterial cell. Examples of bacterial cells that may be used as parental cells include, but are not limited to, Escherichia spp. (e.g., Escherichia coli), Streptococcus spp. (e.g., Streptococcus pyogenes, Streptococcus viridans, Streptococcus pneumoniae), Neisseria spp. (e.g., Neisseria gibirrhoea, Neisseria meningitidis), Corynebacterium spp. (e.g., Corynebacterium diphtheriae), Bacillis spp. (e.g., Bacillis antracis, Bacillis subtilis), Lactobacillus spp., Clostridium spp. (e.g., Clostridium tetani, Clostridium perfringens, Clostridium novyii), Mycobacterium spp. (e.g., Mycobacterium tuberculosis), Shigella spp. (e.g., Shigella flexneri, Shigella dysenteriae), Salmonella spp. (e.g., Salmonella typhi, Salmonella enteritidis), Klebsiella spp. (e.g., Klebsiella pneumoniae), Yersinia spp. (e.g., Yersinia pestis), Serratia spp. (e.g., Serratia marcescens), Pseudomonas spp. (e.g., Pseudomonas aeruginosa, Pseudomonas mallei), Eikenella spp. (e.g., Eikenella corrodens), Haemophilus spp. (e.g., Haemophilus influenza, Haemophilus ducreyi, Haemophilus aegyptius), Vibrio spp. (e.g., Vibrio cholera, Vibrio natriegens), Legionella spp. (e.g., Legionella micdadei, Legionella bozemani), Brucella spp. (e.g., Brucella abortus), Mycoplasma spp. (e.g., Mycoplasma pneumoniae) and Streptomyces spp. (e.g. Streptomyces coelicolor, Streptomyces lividans, Streptomyces albus). In some embodiments, the parental cell is an Escherichia coli cell.

Parental cells are engineered to include a recombineering system, as discussed below, and in some embodiments, a sequence-specific nuclease (e.g., a restriction endonuclease or an RNA-guided nuclease). In some embodiments, a parental cell is also engineered to include a (at least one) selectable marker gene or other heterologous segment. Thus, in some embodiments, methods of the present disclosure include introducing into the parental cell an inducible recombineering system, introducing into the parental cell a nucleic acid encoding a sequence specific nuclease, and/or introducing into a parental cell a selectable marker gene (or other heterologous segment) flanked by homology sequences homologous to sequences flanking a genomic locus of interest.

### Selectable Marker Genes

The methods of the present disclosure, in some embodiments, advantageously avoid recombination crossover events as well as ‘scaring’ of the genome through the use of a selectable marker gene (or other heterologous segment) integrated at genomic loci of interest. Selectable marker genes are genes that confer a trait suitable for artificial selection. Selectable marker genes include genes encoding fluorescent molecules and antibiotic resistance genes. In some embodiments, the selectable marker gene is an antibiotic resistance gene, which confers resistance to a particular antibiotic. It should be understood that the selectable marker genes as used herein may be used, not for the particular trait they confer, but rather for simply containing sequence heterologous to the parental cell. Thus, while a selectable marker gene is used in many embodiments herein, any heterologous gene segment may be used instead of a selectable marker gene.

Antibiotic resistance genes as provided herein may confer resistance to, for example, phleomycin D1 (ZEOCIN™), kanamycin, spectinomycin, streptomycin, ampicillin, carbenicillin, bleomycin, erythromycin, polymyxin B, tetracycline or chloramphenicol. In some embodiments, an antibiotic resistance gene confers resistance to phleomycin D1 (ZEOCIN™). Other antibiotic resistance genes are encompassed by the present disclosure.

Selectable markers genes as provided herein are flanked by homology sequences (also known as homology arms). These homology sequences are contiguous stretches of nucleotide sequences located on each end (5′ end and 3′ end) of a selectable marker gene that are identical or nearly identical to homology sequences flanking a genomic locus of interest in a parental cell. The homology sequences flanking the selectable marker gene recombined (via homologous recombination) with the homology sequences flanking the genomic locus of interest in the parental cell to achieve successful integration of the selectable marker gene into the genome of the parental cell.

The length of a homology sequence for integration of donor DNA may vary. In some embodiments, a homology sequence has a length of longer than 50 nucleotide base pairs (bp). For example, a homology sequence may have a length of at least 60 bp, at least 70 bp, at least 80 bp, at least 90 bp, at least 100 bp, at least 110 bp, at least 120 bp, at least 130 bp, at least 140 bp, at least 150 bp, at least 160 bp, at least 170 bp, at least 180 bp, at least 190 bp, at least 200 bp, at least 210 bp, at least 220 bp, at least 230 bp, at least 240 bp, at least 250 bp, at least 260 bp, at least 270 bp, at least 280 bp, at least 290, at least 300 bp, at least 350 bp, at least 400 bp, at least 450 bp, or at least t 500 bp. In some embodiments, a homology sequence has a length of 100-500 bp, 100-400 bp, 100-300 bp, 100-200 bp, 150-500 bp, 150-400 bp, 150-300 bp, 150-200 bp, 200-500 bp, 200-400 bp, 200-300 bp, 250-500 bp, 250-400 bp, 250-300 bp. In some embodiments, a homology sequence has a length of 250 bp or at least 250 bp. In some embodiments, a homology sequence has a length of longer than 500 bp.

In some embodiments, the parental cell comprises at least two selectable marker genes (or other heterologous segment) integrated genomically and each flanked by homology sequences. For example, the parental cell may include 2, 3, 4 or 5 selectable marker genes.

### Recombineering Systems

Recombineering is a recombination-mediated genetic engineering method based on homologous recombination systems used to combine DNA sequences (e.g., in a specified location and/or order) (see, e.g., Thomason, L., D. L. Court, M. Bubunenko, N. Costantino, H. Wilson, S. Datta & A. Oppenheim, (2007) Recombineering: Genetic engineering in bacteria using homologous recombination. In: Current Protocols in Molecular Biology. Hoboken, N.J.: John Wiley & Sons, Inc., pp. Chapter 1 Unit 16 p. 11-24). Examples of recombineering systems for use in prokaryotic cells as provided herein include the lambda red recombineering system encoding Gam, Exo and Beta proteins and the recombineering system encoding RecE, RecT and Gam proteins. Examples of recombineering systems for use in eukaryotic cells as provided herein include the CRE-lox and Flp-FRT systems. In some embodiments, the recombineering system is inducible. For example, the nucleic acids encoding the required proteins of a recombineering system (e.g., Gam, Exo and Beta) may be operably linked to an inducible promoter. Examples of inducible systems include, but are not limited to, arabinose induction, rhamnose induction, isopropyl β-D-1-thiogalactopyranoside (IPTG) induction and analogues thereof, and anhydrous tetracycline (aTc) induction.

In some embodiments, the inducible recombineering system is integrated genomically in the parental cell.

### Nuclease

Sequence-specific nucleases of the present disclosure are nucleases that cleave nucleic acid at or near a specific sequence within the nucleic acid. Sequence-specific nucleases, as provided herein are used to introduce double-strand breaks at a genomic locus (or loci) of interest to enhance homologous recombination. Non-limiting examples of sequence-specific nucleases include restriction endonucleases (restriction enzymes) and programmable nucleases, such as RNA-guided nucleases, zinc-finger nucleases (ZFNs), and transcription activator-like effector nucleases (TALENs).

Examples of restriction endonucleases include, but are not limited to, EcoRI, EcoRII, BamHI, HindIII, TaqI, NotI, HinFI, Sau3AI, PvuII, SmaI, HaeII, HgaI, AluI, EcoRV, EcoP15I, KpnI, PstI, SacI, SalI, ScaI, SpeI, SphI, StuI, XbaI, AcuI, AlwI, BaeI, BbsI, BbsI-HF, BbvI, BccI, BceAI, BcgI, BciVI, BcoDI, BfuAI, BmrI, BpmI, BpuEI, BsaI, BsaI-HF®, BsaXI, BseRI, BsgI, BsmAI, BsmBI, BsmFI, BsmI, BspCNI, BspMI, BspQI, BsrDI, BsrI, BtgZI, BtsCI, BtsI, BtslMutI, CspCI, EarI, EciI, FauI, FokI, HgaI, HphI, HpyAV, MboII, MlyI, MmeI, MnII, NmeAIII, PleI, SapI, and SfaNI.

Zinc finger nucleases (ZFNs) are a class of engineered DNA-binding proteins that create double-strand breaks in DNA at user-specified locations. Each ZFN includes two functional domains: a DNA-binding domain and a DNA-cleaving domain. The DNA-binding domain is comprised of a chain of two-finger modules, each recognizing a unique hexamer (6 bp) sequence of DNA. Two-finger modules are stitched together to form a zinc finger protein, each with specificity of ≥24 bp. The DNA-cleaving domain is comprised of the nuclease domain of Fok I. When the DNA-binding and DNA-cleaving domains are fused together, a highly-specific pair of ‘genomic scissors’ are created.

Transcription activator-like effector nucleases (TALEN) are restriction enzymes that cleave specific sequences of DNA. TALENs are fusions of transcription activator-like (TAL) proteins and a FokI nuclease. TAL proteins are composed of 33-34 amino acid repeating motifs with two variable positions that have a strong recognition for specific nucleotides

RNA-guided nucleases are based on naturally occurring Type II CRISPR-Cas systems. RNA-guided nuclease systems include a short (e.g., ˜100 nucleotide) guide RNA (gRNA) that uses 20 variable nucleotides at its 5′ end to base pair with a target genomic DNA sequence and a nuclease (e.g., Cas9 endonuclease) that cleaves the target DNA. In some embodiments, the RNA-guided nuclease used as provide herein is a Cas9 nuclease. In other embodiments, the RNA-guided nuclease used as provide herein is a Cpf1 nuclease.

In some embodiments, the methods comprise introducing into the parental cell at least one nucleic acid encoding at least two gRNAs, each targeting a different region of the selectable marker gene, or introducing into the parental cell at least two nucleic acids, each encoding a gRNA that targets (is complementary to) a different region of the selectable marker gene. For example, a gRNA may target a central region of the selectable marker gene and/or a gRNA may target end regions of the selectable marker gene, near the flanking homology sequences.

A sequence-specific nuclease, in some embodiments, may be used to remove a plasmid carrying recombinase, nuclease, and/or gRNA at the end of each assembly cycle (integration of a single donor DNA segment), prior to moving on to the next assembly cycle (to integrate another donor DNA segment). This may be achieved, for example, by introducing into the parental cell a nucleic acid encoding a (at least one) gRNA that targets the nucleic acid (e.g., vector) encoding components of the nuclease system used to facilitate the double-strand breaks in the genome. Thus, in some embodiments, the vector (e.g., plasmid) carrying the nucleic acid encoding the gRNA targeting the genome also carries a nucleic acid encoding a self-targeting and/or gRNA targeting other components of the recombination and nuclease system.

### Donor DNA

The donor DNA segment introduced into a parental cell may be any exogenous DNA segment of interest. In some embodiments, the donor DNA segment is recombinant or synthetic DNA (referred to as “engineered” DNA). Typically, a donor DNA segment is double-stranded. Donor DNA may be generated, for example, by DNA synthesis (e.g., oligonucleotides, GENEBYTES™), PCR, or any other assembly method (e.g., Gibson, yeast assembly, etc.).

The length of a donor DNA segment may vary (e.g., 20 kilobases (kb) to 1000 kb). The methods of the present disclosure are useful, for example, for assembling large DNA segments, thus, in some embodiments, the length of a donor DNA segment is at least 50 kb. For example, the length of a donor DNA segment may be at least 100 kb, at least 150 kb, at least 200 kb, at least 250 kb, at least 300 kb, at least 350 kb, at least 400 kb, at least 450 kb or at least 500 kb. In some embodiments, the length of a donor DNA segment is 50-1000 kb, 50-500 kb, 50-250 kb, 50-200 kb, or 50-150 kb, or 50-100 kb. In some embodiments, the length of the donor DNA segment is less than 50 kb (e.g., 40 kb, 30 kb, 20 kb, or 10 kb).

The methods of the present disclosure, in some embodiments, are used to assemble a complete genome (e.g., a recoded bacterial genome). Thus, in some embodiments, the donor DNA segment is a genomic DNA segment. In some embodiments, the donor DNA segment is a modified (e.g., mutated) genomic segment homologous to a DNA segment (e.g., WT DNA segment) of a parental cell. For example, a DNA segment obtained from a WT E. coli cell may be modified (e.g., recoded) to replace a specific codon. This modified DNA segment may then be used as a donor DNA segment to replace the corresponding WT DNA segment of a WT E. coli parental cell, thus recoding the cell. In some embodiments, the donor DNA segment is a synthetic DNA segment (is synthesized). In some embodiments, the donor DNA segment is a combination of WT DNA and synthetic DNA.

Two DNA segments are considered homologous to each other if they share similar sequences, are obtained from similar positions in a genome, and/or share a similar evolutionary origin. Homologous DNA segments may, but do not necessarily, share the same function. In some embodiments, a donor DNA segment is considered homologous to a genomic DNA segment of a parental cell if the nucleotides sequences of the two segments are at least 80% identical (share at least 80% sequence identity). In some embodiments, a donor DNA segment is considered homologous to a genomic DNA segment of a parental cell if the nucleotides sequences of the two segments are at least 85%, at least 90%, at least 95%, at least 96%, at least 97%, at least 98%, or at last 99% identical to each other.

Donor DNA segments are flanked by homology sequences to facilitate homologous recombination with a genomically integrated selectable marker gene. These homology sequences are contiguous stretches of nucleotide sequences located on each end (5′ end and 3′ end) of a donor DNA segment that are identical or nearly identical to homology sequences flanking selectable marker gene genomically integrated in a parental cell. The homology sequences flanking the donor DNA segment recombined (via homologous recombination) with the homology sequences flanking the selectable marker gene in the parental cell to achieve successful integration of the donor DNA segment into the genome of the parental cell, replacing the selectable marker gene.

In some embodiments, the donor DNA segment is a modified genomic segment homologous to a DNA segment of the parental cell that has been replaced by the selectable marker gene. That is, the donor DNA segment may contain at least one (e.g., 2, 3, 4 or more) mutation (e.g., point mutation, insertion and/or deletion) relative to a corresponding wild-type DNA segment of the parental cell.

In some embodiments, the methods comprises introducing into the parental cell (e.g., simultaneously/in parallel) at least two donor DNA segments, each flanked by homology sequences, wherein each homology sequence of a donor DNA segment is homologous to a homology sequence of one of the at least two selectable marker genes. For example, the methods may comprise introducing into the parental cell (e.g., simultaneously/in parallel) 2, 3, 4 or 5 different donor DNA segments, each, for example, located on a separate vector (e.g., plasmid).

### Exemplary Applications

In some embodiments, the methods of the present disclosure are used to assemble a recoded genome, for example an E. coli genome.

In some embodiments, the methods of the present disclosure are used to integrate large (e.g., longer than 50 kb) DNA segments into the genome, including whole pathways and synthetic DNA.

In some embodiments, the methods of the present disclosure are used to exchange large DNA segments between plasmids.

In some embodiments, the methods of the present disclosure are used for multiplexed integration of large-size DNA libraries, for example recoded segment libraries.

### Compositions and Kits

In some embodiments, the present disclosure provides compositions and/or kits comprising (a) a vector comprising a selectable marker gene flanked by multiple cloning sites, or flanked by homology sequences homologous to sequences flanking a genomic locus of interest; (b) a vector comprising an inducible recombineering system; and (c) a vector comprising a nucleic acid encoding a sequence-specific nuclease. In some embodiment, the sequence-specific nuclease is a restriction endonuclease. In some embodiment, the sequence-specific nuclease is a RNA-guided nuclease. Thus, in some embodiments, the vector of (c) further comprises a nucleic acid encoding a guide RNA (gRNA) targeting the selectable marker gene.

In other embodiments, the present disclosure provide compositions and/or kits comprising (a) a vector comprising a selectable marker gene flanked by multiple cloning sites, or flanked by homology sequences homologous to sequences flanking a genomic locus of interest; (b) a vector comprising an inducible recombineering system; and (c) a nuclease. In some embodiment, the sequence-specific nuclease is a restriction endonuclease. In some embodiment, the sequence-specific nuclease is a RNA-guided nuclease. Thus, in some embodiments, the compositions and/or kits further comprise (d) a vector comprising a nucleic acid encoding a guide RNA (gRNA) targeting the selectable marker gene.

In some embodiments, the compositions and/or kits further comprise a donor DNA segment flanked by homology sequences homologous to the homology sequences of (a). In some embodiments, the donor DNA segment has a length of at least 50 nucleotide base pairs.

### REFERENCES

1. N. Ostrov et al., Design, synthesis, and testing toward a 57-codon genome. Science. 353, 819-822 (2016).
2. K. Wang et al., Defining synonymous codon compression schemes by genome recoding. Nature (2016), doi:10.1038/nature20124.
3. H. H. Wang et al., Programming cells by multiplex genome engineering and accelerated evolution. Nature. 460, 894-898 (2009).
4. M. C. Bassalo et al., Rapid and Efficient One-Step Metabolic Pathway Integration in E. coli. ACS Synth. Biol. 5, 561-568 (2016).
5. J. Zhou, R. Wu, X. Xue, Z. Qin, CasHRA (Cas9-facilitated Homologous Recombination Assembly) method of constructing megabase-sized DNA. Nucleic Acids Res. 44, e124 (2016).
6., doi:10.1101/130088.
7. J. C. van Kessel, G. F. Hatfull, Recombineering in Mycobacterium tuberculosis. Nat. Methods. 4, 147-152 (2007).
8. J. I. Katashkina et al., Use of the λ Red-recombineering method for genetic engineering of Pantoea ananatis. BMC Mol. Biol. 10, 34 (2009).
9. W. W. Metcalf et al., Conditionally replicative and conjugative plasmids carrying lacZ alpha for cloning, mutagenesis, and allele replacement in bacteria. Plasmid. 35, 1-13 (1996).

### EXAMPLES

### Example 1. Delivery of Donor DNA by Conjugation

This Example demonstrates delivery of donor DNA (60 kb plasmid) into parental cells by way of conjugation. The F-Cas plasmid (W. W. Metcalf et al. Plasmid. 35, 1-13 (1996)), which has the required machinery to conjugate F-plasmids, was used to conjugate a plasmid carrying recoded DNA from Top10 E. coli into EcM2.1 or MG1665 E. coli. Recoded segments 14 and 83 (W. W. Metcalf et al. (1996)) were used, each 50 kb in size with a total plasmid size of 60 kb.

First, F-cas was conjugated into Top10 containing a recoded segment. The plasmid from Top10 to EcM2.1 was then conjugated and plated on selective media (Carb selection for acceptor strain EcM2.1, spectinomycin selection for recoded segment plasmid). Conjugation of two recoded plasmids into EcM2.1 (segments 14 and 83) was successfully demonstrated.

### Example 2. Integration of Recoded Segments at the Correct Genomic Locus

This Example demonstrates scarless integration of a recoded segment (50 kb) into the E. coli (parental) genome, using lambda-red and Cas9. The parental strain carries the lambda-red system on a plasmid, and donor DNA (introduced by conjugation, see Example 1). Homologous sequences in the parent strain were also removed prior to integration (the genomic sequence corresponding to recoded segment). The recipient strain thus carries a ZEOCIN™ resistance gene (selectable marker) at the desired loci for genomic integration, flanked by 250 bp of overlapping sequence with donor DNA.

Cells were grown with selection for lambda-red and donor DNA, as well as induction (arabinose) for expression of red recombinase. A plasmid carrying Cas9 nuclease and a gRNA targeting the ZEOCIN™ resistance gene were transform. By targeting a selectable marker gene in the genome (rather than a specific E. coli genome sequence) this general-use Cas9 plasmid can be recycled and used with any recoded segment, regardless of targeted genome loci.

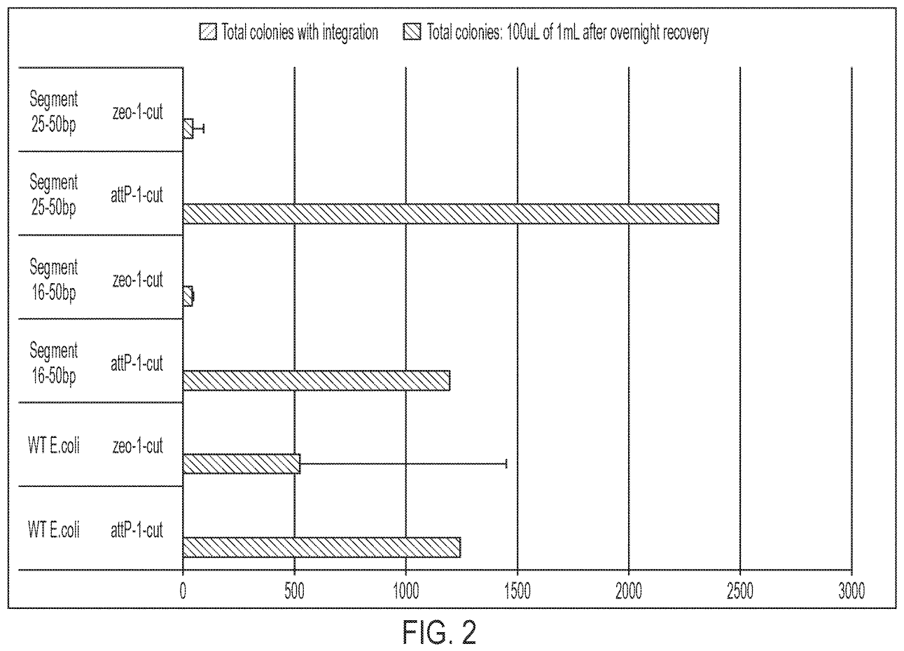

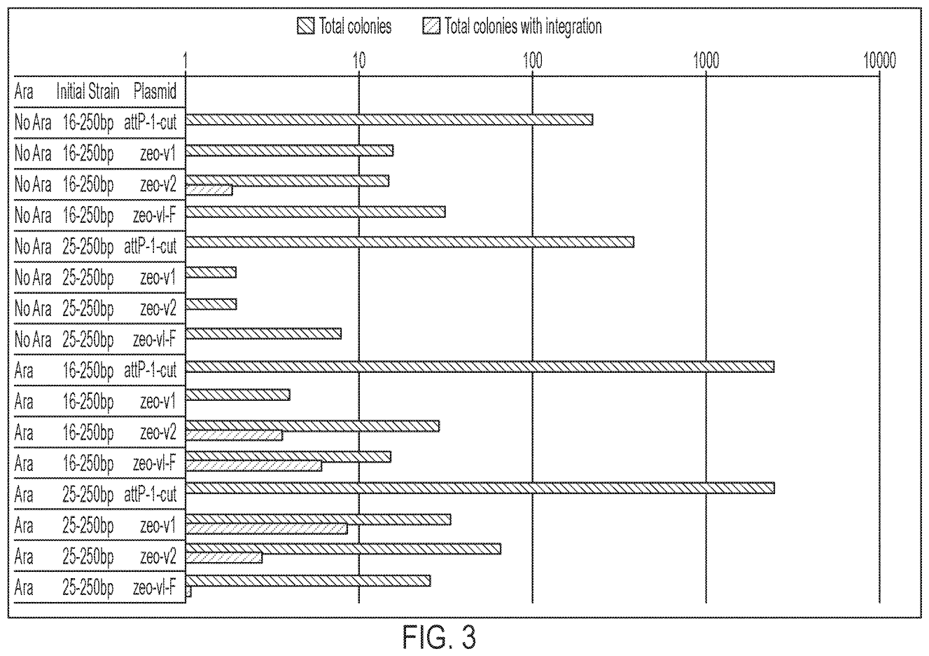

Cells were recovered in LB media and then plated to select for Cas9 plasmid only (carbenicillin). The resulting colonies were screened by PCR and sequencing to identify integration of donor DNA and loss of ZEOCIN™ resistance gene at the desired genomic location. Cells carrying Cas9 plasmid should actively cut that ZEOCIN™ resistance gene in the parental genome. FIGS. 2 and 3 provide data showing experimental results for this example. We found that a larger homology sequence (250 bp) is preferred.

### Example 3. Hierarchical Assembly of a Synthetic Genome (e.g., r E. Coli-57)

Recoded segments are gradually added, in a hierarchical manner, to construct a fully recoded genome. Below, ‘I-rc’ represents any recoded segment on a plasmid, and ‘I-wt’ represent the corresponding wild-type (wt) genes in the genome (to be replaced by a recoded segment).
1. Parental strain contains recombinase expression system.
2. Introduce recoded segment (i-rc) on a plasmid (e.g., by conjugation, transformation, or transduction) into parental strain.
3. Delete i-wt using homologous recombination of PCR cassette. The cassette contains a selectable marker gene (for example: zeoR or kanR) flanked by 250 bp of homology with i-rc.
4. Induce recombination: transform plasmid carrying Cas9 and gRNA targeted to cut the selectable marker gene. Cutting induces recombination to integrate i-rc at the designated loci in the genome.
5. Reset step: remove any remaining plasmids not needed for next cycle (gRNA plasmid and previous segment plasmid, if remains. This step can be done using negative selection (for example TolC positive/negative selection for plasmids) or using a gRNA that targets the plasmid backbone for cutting by Cas9.
6. Conjugate next segment into recipient (segment i+1).
7. Iterate.

To sequentially integrate multiple segments, this method uses a recombinase, single Cas9-gRNA system, and a selectable marker (e.g., zeoR)-deletion cassette for each donor DNA segment. At the end of each cycle, the gRNA component is removed to avoid interruption with the incoming selectable marker (e.g., zeoR)-deletion cassette in the next cycle. This linear assembly process can be used to sequentially add recoded segments for full chromosome recoding.

What is claimed is:

1. A method, comprising:
(a) introducing into a parental cell a donor DNA segment flanked by first homology sequences, wherein the parental cell comprises (i) a selectable marker gene integrated genomically and flanked by second homology sequences homologous to the first homology sequences, and (ii) an inducible recombineering system;
(b) introducing into the parental cell sequence-specific nuclease or a nucleic acid encoding a sequence-specific nuclease targeting the selectable marker gene; and
(c) inducing expression of the inducible recombineering system.

2. A method, comprising:
(a) introducing into a parental cell a donor DNA segment flanked by first homology sequences, wherein the parental cell comprises (i) a selectable marker gene integrated genomically and flanked by second homology sequences homologous to the first homology sequences, and (ii) an inducible recombineering system;
(b) introducing into the parental cell (i) a RNA-guided nuclease or a nucleic acid encoding a RNA-guided nuclease and (ii) at least one nucleic acid encoding at least one guide RNA (gRNA) targeting the selectable marker gene; and
(c) inducing expression of the inducible recombineering system.

3. The method of claim 1 or 2 further comprising assaying the parental cell for the presence of the nuclease.

4. The method of any one of claims 1-3, wherein step (c) is performed before step (b).

5. The method of any one of claims 1-4, wherein the donor DNA segment has a length of at least 50 kilobases.

6. The method of any one of claims 1-5, wherein the donor DNA segment is a modified genomic segment homologous to a DNA segment of the parental cell that has been replaced by the selectable marker gene.

7. The method of any one of claims 1-6, wherein each of the homology sequences has a length of greater than 50 nucleotide base pairs.

8. The method of claim 7, wherein each of the homology sequences has a length of at least 100 nucleotide base pairs.

9. The method of claim 8, wherein each of the homology sequences has a length of at least 250 nucleotide base pairs.

10. The method of any one of claims 1-9, wherein the selectable marker gene is an antibiotic resistance gene.

11. The method of claim 9, wherein the antibiotic resistance gene confers resistance to phleomycin D1 (ZEOCIN™), kanamycin, spectinomycin, streptomycin, ampicillin, carbenicillin, bleomycin, erythromycin, polymyxin B, tetracycline and chloramphenicol.

12. The method of claim 11, wherein the antibiotic resistance gene confers resistance to phleomycin D1 (ZEOCIN™).

13. The method of any one of claim 1 or 4-12, wherein the sequence specific nuclease is a restriction endonuclease.

14. The method of any one of claim 1 or 4-12, wherein the sequence specific nuclease is a programmable nuclease.

15. The method of any one of claims 2-12, wherein the RNA-guided nuclease is selected from Cas9 nuclease and Cpf1 nuclease.

16. The method of claim 15, wherein the RNA-guided nuclease is Cas9 nuclease.

17. The method of any one of claims 1-16, wherein the inducible recombineering system is selected from an inducible recombineering system encoding Gam, Exo and Beta proteins and an inducible recombineering system encoding RecE and RecT proteins.

18. The method of claim 17, wherein the inducible recombineering system is an inducible recombineering system encoding Gam, Exo and Beta proteins.

19. The method of any one of claims 1-18, wherein the inducible recombineering system is integrated genomically in the parental cell.

20. The method of any one of claims 2-19 further comprising introducing into the parental cell at least one nucleic acid encoding at least one gRNA targeting the at least one nucleic acid of (b)(ii).

21. The method of any one of claims 2-20, wherein step (b)(ii) comprises introducing into the parental cell at least one nucleic acid encoding at least two gRNAs, each targeting a different region of the selectable marker gene, or introducing into the parental cell at least two nucleic acids, each encoding a gRNA that targets a different region of the selectable marker gene.

22. The method of any one of claims 1-21 further comprising repeating steps (a)-(c) using a DNA segment having a sequence that is different from the DNA segment of step (a).

23. The method of claim 22 further comprising repeating steps (a)-(c) multiple times, each time using a DNA segment having a sequence that is different from any other DNA segment introduced into the parental cell.

24. The method of any one of claims 1-23 further comprising, prior to step (a):
introducing into the parental cell the selectable marker gene flanked by homology sequences homologous to sequences flanking a genomic locus of interest; and/or
introducing into the parental cell the inducible recombineering system.

25. The method of any one of claims 1-24, wherein the parental cell of comprises at least two selectable marker genes integrated genomically and each flanked by homology sequences.

26. The method of claim 25 further comprising introducing into the parental cell at least two donor DNA segments, each flanked by homology sequences, wherein each homology sequence of a donor DNA segment is homologous to a homology sequence of one of the at least two selectable marker genes.

27. A method, comprising:
(a) introducing into a parental cell a donor DNA segment flanked by first homology sequences, wherein the parental cell comprises (i) a selectable marker gene integrated genomically and flanked by second homology sequences homologous to the first homology sequences, (ii) an inducible recombineering system, and (iii) a nucleic acid encoding a sequence-specific nuclease; and
(c) inducing expression of the inducible recombineering system.

28. A method, comprising:
(a) introducing into a parental cell a donor DNA segment flanked by first homology sequences, wherein the parental cell comprises (i) a selectable marker gene integrated genomically and flanked by second homology sequences homologous to the first homology sequences, (ii) an inducible recombineering system, and (iii) a nucleic acid encoding a RNA-guided nuclease;
(b) introducing into the parental cell a nucleic acid encoding a guide RNA (gRNA) targeting the selectable marker gene; and
(c) inducing expression of the inducible recombineering system.

29. The method of claim 27 or 28 further comprising assaying the parental cell for the presence of the nuclease.

30. The method of claim 28 or 29, wherein step (c) is performed before step (b).

31. The method of any one of claims 27-30, wherein the donor DNA segment has a length of at least 50 kilobases.

32. The method of any one of claims 27-31, wherein the donor DNA segment is a modified genomic segment homologous to a DNA segment of the parental cell that has been replaced by the selectable marker gene.

33. The method of any one of claims 27-32, wherein each of the homology sequences has a length of greater than 50 nucleotide base pairs.

34. The method of claim 33, wherein each of the homology sequences has a length of at least 100 nucleotide base pairs.

35. The method of claim 34, wherein each of the homology sequences has a length of at least 250 nucleotide base pairs.

36. The method of any one of claims 27-35, wherein the selectable marker gene is an antibiotic resistance gene.

37. The method of claim 35, wherein the antibiotic resistance gene confers resistance to phleomycin D1 (ZEOCIN™), kanamycin, spectinomycin, streptomycin, ampicillin, carbenicillin, bleomycin, erythromycin, polymyxin B, tetracycline and chloramphenicol.

38. The method of claim 37, wherein the antibiotic resistance gene confers resistance to phleomycin D1 (ZEOCIN™).

39. The method of any one of claim 27 or 30-38, wherein the sequence specific nuclease is a restriction endonuclease.

40. The method of any one of claim 27 or 30-38, wherein the sequence specific nuclease is a programmable nuclease.

41. The method of any one of claims 28-38, wherein the nucleic acid encoding the RNA-guided nuclease is integrated genomically in the parental cell.

42. The method of claim 41, wherein expression of the nucleic acid encoding the RNA-guided nuclease is inducible.

43. The method of claim 42 wherein the nucleic acid encoding the RNA-guided nuclease is operably linked to an inducible promoter.

44. The method of any one of claims 28-43, wherein the RNA-guided nuclease is selected from Cas9 nuclease and Cpf1 nuclease.

45. The method of claim 44, wherein the RNA-guided nuclease is Cas9 nuclease.

46. The method of any one of claims 27-45, wherein the inducible recombineering system is selected from an inducible recombineering system encoding Gam, Exo and Beta proteins and an inducible recombineering system encoding RecE and RecT proteins.

47. The method of claim 46, wherein the inducible recombineering system is an inducible recombineering system encoding Gam, Exo and Beta proteins.

48. The method of any one of claims 27-47, wherein the inducible recombineering system is integrated genomically in the parental cell.

49. The method of any one of claims 28-48 further comprising introducing into the parental cell at least one nucleic acid encoding at least one gRNA targeting the at least one nucleic acid of (b).

50. The method of any one of claims 28-49, wherein step (b)(ii) comprises introducing into the parental cell at least one nucleic acid encoding at least two gRNAs, each targeting a different region of the selectable marker gene, or introducing into the parental cell at least two nucleic acids, each encoding a gRNA that targets a different region of the selectable marker gene.

51. The method of any one of claims 27-50 further comprising repeating steps (a)-(c) using a DNA segment having a sequence that is different from the DNA segment of step (a).

52. The method of claim 51 further comprising repeating steps (a)-(c) multiple times, each time using a DNA segment having a sequence that is different from any other DNA segment introduced into the parental cell.

53. The method of any one of claims 27-52 further comprising, prior to step (a):
introducing into the parental cell the selectable marker gene flanked by homology sequences homologous to sequences flanking a genomic locus of interest; and/or
introducing into the parental cell the inducible recombineering system.

54. The method of any one of claims 27-53, wherein the parental cell of comprises at least two selectable marker genes integrated genomically and each flanked by homology sequences.

55. The method of claim 54 further comprising introducing into the parental cell at least two donor DNA segments, each flanked by homology sequences, wherein each homology sequence of a donor DNA segment is homologous to a homology sequence of one of the at least two selectable marker genes.

56. An engineered cell comprising
(a) a selectable marker gene genomically integrated and flanked by homology sequences;
(b) an inducible recombineering system;
(c) a RNA-guided nuclease or a nucleic acid encoding a RNA-guided nuclease; and
(d) a nucleic acid encoding at least one guide RNA (gRNA) targeting the selectable marker gene.

57. The engineered cell of claim 56, further comprising a donor DNA segment flanked by homology sequences homologous to the homology sequences of (a).

58. The engineered cell of claim 57, wherein the donor DNA segment has a length of at least 50 kilobases.

59. The engineered cell of any one of claims 56-58, wherein the donor DNA segment is a modified genomic segment homologous to a DNA segment of the engineered cell that has been replaced by the selectable marker gene.

60. The engineered cell of any one of claims 56-59, wherein each of the homology sequences has a length of greater than 50 nucleotide base pairs.

61. The engineered cell of claim 60, wherein each of the homology sequences has a length of at least 100 nucleotide base pairs.

62. The engineered cell of claim 61, wherein each of the homology sequences has a length of at least 250 nucleotide base pairs.

63. The engineered cell of any one of claims 56-62, wherein the selectable marker gene is an antibiotic resistance gene.

64. The engineered cell of claim 63, wherein the antibiotic resistance gene confers resistance to phleomycin D1 (ZEOCIN™), kanamycin, spectinomycin, streptomycin, ampicillin, carbenicillin, bleomycin, erythromycin, polymyxin C, tetracycline and chloramphenicol.

65. The engineered cell of claim 64, wherein the antibiotic resistance gene confers resistance to phleomycin D1 (ZEOCIN™).

66. The engineered cell of any one of claims 56-65, wherein the nucleic acid encoding the RNA-guided nuclease is integrated genomically in the engineered cell.

67. The engineered cell of claim 66, wherein expression of the nucleic acid encoding the RNA-guided nuclease is inducible.

68. The engineered cell of claim 67 wherein the nucleic acid encoding the RNA-guided nuclease is operably linked to an inducible promoter.

69. The engineered cell of any one of claims 56-68, wherein the RNA-guided nuclease is selected from Cas9 nuclease and Cpf1 nuclease.

70. The engineered cell of claim 69, wherein the RNA-guided nuclease is Cas9 nuclease.

71. The engineered cell of any one of claims 56-70, wherein the inducible recombineering system is selected from an inducible recombineering system encoding Gam, Exo and Beta proteins and an inducible recombineering system encoding RecE and RecT proteins.

72. The engineered cell of claim 71, wherein the inducible recombineering system is an inducible recombineering system encoding Gam, Exo and Beta proteins.

73. The engineered cell of any one of claims 56-72, wherein the inducible recombineering system is integrated genomically in the engineered cell.

74. The engineered cell of any one of claims 56-73, wherein the engineered cell of comprises at least two selectable marker genes integrated genomically and each flanked by homology sequences.

75. The engineered cell of claim 74 further comprising at least two donor DNA segments, each flanked by homology sequences, wherein each homology sequence of a donor DNA segment is homologous to a homology sequence of one of the at least two selectable marker genes.

76. A kit comprising:
(a) a vector comprising a selectable marker gene flanked by multiple cloning sites, or flanked by homology sequences homologous to sequences flanking a genomic locus of interest;
(b) a vector comprising an inducible recombineering system; and
(c) a vector comprising a nucleic acid encoding a RNA-guided nuclease and a guide RNA (gRNA) targeting the selectable marker gene.

77. A kit comprising:
(a) a vector comprising a selectable marker gene flanked by multiple cloning sites, or flanked by homology sequences homologous to sequences flanking a genomic locus of interest;
(b) a vector comprising an inducible recombineering system;
(c) a RNA-guided nuclease; and
(d) a vector comprising a nucleic acid encoding a guide RNA (gRNA) targeting the selectable marker gene.

78. The kit of claim 76, further comprising a donor DNA segment flanked by homology sequences homologous to the homology sequences of (a).

79. The kit of claim 78, wherein the donor DNA segment has a length of at least 50 kilobases.

80. The kit of claim 78 or 79, wherein the donor DNA segment is a modified genomic segment homologous to a DNA segment of the kit that has been replaced by the selectable marker gene.

81. The kit of any one of claims 76-80, wherein each of the homology sequences has a length of greater than 50 nucleotide base pairs.

82. The kit of claim 81, wherein each of the homology sequences has a length of at least 100 nucleotide base pairs.

83. The kit of claim 81, wherein each of the homology sequences has a length of at least 250 nucleotide base pairs.

84. The kit of any one of claims 76-83, wherein the selectable marker gene is an antibiotic resistance gene.

85. The kit of claim 84, wherein the antibiotic resistance gene confers resistance to phleomycin D1 (ZEOCIN™), kanamycin, spectinomycin, streptomycin, ampicillin, carbenicillin, bleomycin, erythromycin, polymyxin D, tetracycline and chloramphenicol.

86. The kit of claim 85, wherein the antibiotic resistance gene confers resistance to phleomycin D1 (ZEOCIN™).

87. The kit of any one of claims 76-86, wherein the nucleic acid encoding the RNA-guided nuclease is operably linked to an inducible promoter.

88. The kit of any one of claims 76-87, wherein the RNA-guided nuclease is selected from Cas9 nuclease and Cpf1 nuclease.

89. The kit of claim 88, wherein the RNA-guided nuclease is Cas9 nuclease.

90. The kit of any one of claims 76-89, wherein the inducible recombineering system is selected from an inducible recombineering system encoding Gam, Exo and Beta proteins and an inducible recombineering system encoding RecE and RecT proteins.

91. The kit of claim 90, wherein the inducible recombineering system is an inducible recombineering system encoding Gam, Exo and Beta proteins.

92. The kit of any one of claims 76-91 further comprising transformation reagents.

93. The kit of any one of claims 76-92, wherein the vector of (a), (b), (c) and/or (d) is a plasmid.

94. The kit of claim 93, wherein the plasmid is a conjugative plasmid.
